(19) United States
(12) Patent Application Publication
(10) Pub. No.: US 2023/0126610 A1
(43) Pub. Date: Apr. 27, 2023

(54) COMPOSITION FOR PREVENTING DETERIORATION IN COGNITIVE FUNCTION AND/OR IMPROVING COGNITIVE FUNCTION

(71) Applicants: PHARMA FOODS INTERNATIONAL CO., LTD., Kyoto (JP); MITSUBISHI CORPORATION LIFE SCIENCES LIMITED, Tokyo (JP)

(72) Inventors: Atsushi YAMATSU, Kyoto (JP); Utano NAKAMURA, Kyoto (JP); Tomoko SAKATA, Kyoto (JP); Mujo KIM, Kyoto (JP); Yasuhiro YOKOYAMA, Tokyo (JP)

(21) Appl. No.: 17/910,580

(22) PCT Filed: Mar. 8, 2021
(86) PCT No.: PCT/JP2021/009017

§ 371 Date: Sep. 9, 2022

(30) Foreign Application Priority Data
Mar. 9, 2020 (JP) 2020-039897

(51) Int. Cl. A61K 31/197; A23L 33/175; A61P 25/28

(52) U.S. Cl. CPC A61K 31/197; A23L 33/175; A61P 25/28

(57) ABSTRACT

Provided is a novel composition for preventing the deterioration in a cognitive function or improving the cognitive function in a subject. A composition for preventing the deterioration in a cognitive function and/or improving the cognitive function in a subject, the composition containing γ-aminobutyric acid.

### TECHNICAL FIELD

The present disclosure is directed to a composition for suppressing reduction in cognitive function and/or improving cognitive function.

### BACKGROUND ART

Due to the rapid aging of population in recent years, recognition regarding the health of elderly people and approach to health are greatly changing. Specifically, in the past, the focus was particularly on extension of healthy-life expectancy, where the health of elderly people referred to the health of the body, but in recent years, the focus is not only on the health of the body, but also on the health of the brain for living a comfortable life after retirement.

The focus is especially on disorders of cognitive function including dementia, wherein dementia would cause secondary problems such as wandering and traffic accidents and problems involving others such as nursing fatigue. In addition, there are more elderly people who fall under mild cognitive impairment (MCI), which is a state of being forgetful and having a reduced cognitive function due to aging but having no hindrance to daily life. It is also considered that about one in four elderly people falls under this potential dementia. Mild cognitive impairment is also considered a state of being one step away from dementia, wherein if left untreated, the symptoms would worsen in the future, leading to dementia.

Most dementia conditions are progressive diseases, wherein the progression of a symptom can be delayed, but complete cure is not possible in today's medical practice. Thus, as a countermeasure for dementia, it is very important to achieve early discovery/diagnosis to stop the symptoms before dementia develops.

### SUMMARY OF INVENTION

### Technical Problem

The present invention was invented in view of such a circumstance, and for the purpose of providing a new composition for suppressing reduction in cognitive function or improving the cognitive function in a subject.

### Solution to Problem

The inventors of the present invention carried out several earnest studies to solve the above-described problem and as a result confirmed that γ-aminobutyric acid (GABA) has the effect of improving cognitive function of a human and achieved completion of the present invention by carrying out further studies based on said finding.

Therefore, according to the major points of the present disclosure, the following inventions are provided.

(1) A composition for suppressing reduction in cognitive function and/or improving the cognitive function in a subject, comprising γ-aminobutyric acid.
(2) The composition of the item above, wherein the cognitive function comprises reasoning.
(3) The composition of any one of the items above, wherein the cognitive function comprises working memory.
(4) The composition of any one of the items above, wherein the cognitive function comprises sustained attention.
(5) The composition of any one of the items above, wherein the reasoning, the working memory, or the sustained attention is measured by Cognitrax.
(6) The composition of any one of the items above, wherein the cognitive function comprises spatial cognition.
(7) The composition of any one of the items above, wherein the cognitive function comprises memory.
(8) The composition of any one of the items above, wherein the spatial cognition or the memory is measured by RBANS neuropsychological test.
(9) The composition of any one of the items above, which is for improving physical function.
(10) The composition of any one of the items above, which is for improving vitality.
(11) The composition of any one of the items above, which is for improving mental health.
(12) The composition of any one of the items above, wherein the physical function, the vitality, or the mental health is measured by SF-36.
(13) The composition of any one of the items above, which is for suppressing reduction in cognitive function and/or improving the cognitive function in a subject by increasing an insulin-like growth factor-1 (IGF-1) concentration in blood.
(14) The composition of any one of the items above, wherein a content of the γ-aminobutyric acid is about 10 to about 1000 mg.
(15) The composition of any one of the items above, wherein an ingestion amount of the γ-aminobutyric acid is about 10 to about 1000 mg per day.
(16) The composition of any one of the items above, which is ingested every day for at least four weeks.
(17) The composition of any one of the items above, wherein the subject is a healthy person.
(18) The composition of any one of the items above, wherein the subject has a Mini Mental State Examination (QAMSE) score of 24 or higher.
(19) The composition of any one of the items above, which is orally ingested.
(20) The composition of any one of the items above, which is a drinking/eating product, a food additive, a quasi-drug, or a medicament.

The characteristics and significant actions/effects of the present disclosure that are not described above would be clear to those skilled in the art by seeing the following embodiment section and drawings of the invention.

One embodiment and example of the present disclosure are explained below while referring to the drawings.

As described above, one embodiment of the present disclosure is a composition for suppressing reduction in cognitive function and/or improving the cognitive function in a subject.

γ-aminobutyric acid (GABA, 4-aminobutyric acid) has been recently receiving attention as an amino acid that is widely distributed in the natural world and can be added to food without altering its taste. GABA is known to be a suppressive neurotransmitter, many of which exist in a central nervous system of a mammal, suppressing excessive secretion of excitatory neurotransmitter to relieve excitation of a nerve, thus exerting a relaxing effect or anti-stress action. In addition, it is known to have various physiological activities such as lowering blood pressure, reducing blood cholesterol concentration, relieving stress, reducing the feeling of fatigue, improving quality of sleep and suppressing reduction in immunity. GABA is also found in vegetables, grain and human bodies, and can thus easily be added to food. Chocolates and many supplements comprising GABA are sold.

Since GABA is an amino acid widely distributed in the natural world such as in vegetables and grain, the origin or the like of GABA is not particularly limited as long as it can be used in drinks or food in one embodiment of the present disclosure. For example, an extract, a purified product, or the like of a plant comprising GABA may be used, or it is possible to carry out preparation from a fermented product obtained by adding glutamic acid decarboxylase, a microorganism having said enzyme such as lactic acid bacteria, or the like to a raw material comprising glutamic acid. A product comprising GABA or GABA of a commercially available product can also be a raw material of the composition of the present disclosure in the scope that does not harm the effect caused by the composition of the present disclosure.

In one embodiment of the present disclosure, cognitive function in a subject can be measured by a cognitive function examination and a neuropsychological test. The cognitive function examination and the neuropsychological test may be either of a question-type examination and an observation-type examination, which can include, for example, Cognitrax test (Cognitrax), RBANS neuropsychological test (RBANS), Hasegawa Dementia Scale-Revised (HDS-R), Mini-Cog, MoCA, Dementia Assessment Sheet in Community-based Integrated Care System (DASC-21), Mini Mental State Examination (MMSE), ABC-Dementia Scale (ABC-DS), Clock Drawing Test (CDT), FAB (Frontal Assessment Battery), ADAS (Alzheimer's Disease Assessment Scale), CDR (Clinical Dementia Rating), Wechsler Adult Intelligence Scale and the like. Although the cognitive function examination and the neuropsychological test are not particularly limited to the above as long as it is possible to understand the intellectual function or cognitive function in the subject, Cognitrax test and RBANS neuropsychological test are preferred. While reduction in cognitive function is generally likely to be considered equivalent to reduction in memory, the actual cognitive function of a human consists of composite association of various abilities such as memory, thought, understanding, calculation, learning, language and judgement. Therefore, when a cognitive function examination and a neuropsychological test are used to assess the effect of a tested food on a human cognitive function, it is preferable to be able to assess a plurality of domains or items of cognitive function.

A Cognitrax test is known as such an examination that can assess a plurality of domains or items of cognitive function. The Cognitrax test is a cognitive function examination service practiced on the Web based on the cognitive function examination technique developed by CNS Vital Signs in the United States. In the Cognitrax test, among a total of 10 types of tests, which are verbal memory test, visual memory test, finger tapping test, symbol digit coding test (SDC test), Stroop test, shifting attention test, continuous performance test, perception of emotion test, reasoning test and four part continuous performance test, a test is carried out in accordance with the purpose of the examination to digitize the examination result regarding each of a total of 16 types of items at most, which are neurocognition index, composite memory, verbal memory, visual memory, psychomotor speed, reaction time, complex attention, cognitive flexibility, processing speed, executive function, social acuity, non-verbal reasoning, working memory, continuous attention, simple attention and motor speed.

In addition, the RBANS neuropsychological test (RBANS: Repeatable Battery for the Assessment of Neuropsychological Status) is one of the neuropsychological examinations that have been standardized in the United States, which is a neuropsychological test exercise (examination battery) that takes a short time (about 30 minutes) and can be repeatedly assessed. The RBANS neuropsychological test can assess five cognitive domains, which are immediate memory, visual spatial/constructional, language, attention and delayed memory.

In one embodiment of the present disclosure, the cognitive function of which reduction is suppressed by the composition of the present disclosure and/or the cognitive function that is improved by the composition of the present disclosure includes reasoning, working memory, sustained attention, spatial cognition, and memory, but is not particularly limited as long as the cognitive function can be measured by the above-mentioned cognitive function examination and neuropsychological test. In this regard, “reasoning” is the ability to logically think based on a so-called logic, including, for example, the non-verbal reasoning assessed by the Cognitrax test. In addition, “working memory” is the ability of operation while temporarily maintaining information, including, for example, the working memory assessed by the Cognitrax test. “Sustained attention” is the ability of continuously maintaining attention to a matter, including, for example, the continuous attention assessed by the Cognitrax test. “Spatial cognition” is the ability of quickly and accurately recognizing the state or relationship of objects occupying a three-dimensional space such as the position, direction, pose, size, shape, gap and the like of the objects, including, for example, the visual spatial/constructional ability assessed by the RBANS neuropsychological test. “Memory” is the ability of memorizing a matter, including, for example, the delayed playback assessed by the RBANS neuropsychological test.

Reduction in the cognitive function assessed by such cognitive function examination and neuropsychological test is known to lead to reduction in the quality of life (QOL). For example, reduction in performance speed or execution function leads to difficulty in taking actions or counter measurements in order, and reduction in continuous attention or simple attention leads to not being able to concentrate on one matter or leads to difficulty in simultaneous performance of a plurality of matters. In one embodiment of the present disclosure, the composition of the present disclosure can also improve such disadvantages in everyday life through improvement in the cognitive function.

SF-36 is known as a QOL assessment method associated with health, wherein SF-36 (MOS 36-Item Short-Form Health Survey) is a self-report type health state questionnaire that can rate eight health concepts (physical function, everyday role function (physical), body pain, general health, vitality, social function, everyday role function (mental) and mental health) as scores. A high score of each health concept (subscale) in SF-36 is interpreted as shown below.
Physical function . . . Capable of carrying out every type of activity including vigorous activity.
Everyday role function (physical) . . . There was no physical problem when carrying out work or normal activity during the past month.
Body pain . . . There was no body pain at all during the past month and usual work was not interfered by body pain at all.
General health . . . Health state is very good.
Vitality . . . Always full of energy during the past month.
Social life function . . . The usual social interactions with family, friends, neighbors and other companions were not at all interfered by any physical or psychological reason.
Everyday role function (mental) . . . There was no psychological problem when carrying out work or normal activity during the past month.
Mental health . . . The subject was in a calm, happy and peaceful mood during the past month.

While there are still many unclear points regarding the biochemical mechanism regarding reduction in cognitive function, the IGF-1 (insulin-like growth factor-1) level in blood is known to be reduced by aging. Thus, it is indicated that there is a possibility of this reduction in IGF-1 in blood causing neurodegeneration or causing reduction in cognitive function. In one embodiment of the present disclosure, the composition of the present disclosure can be a composition for suppressing reduction in cognitive function and/or improving the cognitive function through elevation of the IGF-1 concentration in blood.

In one embodiment of the present disclosure, the content of GABA in the composition of the present disclosure is not particularly limited as long as the concentration or weight is enough to achieve the effect of the composition of the present disclosure in accordance with the method of administration thereof or the like. The content of GABA in the composition of the present disclosure is preferably about 10 to about 1000 mg, wherein the lower limit of the content may be, for example, about 10 mg or greater, about 30 mg or greater, about 50 mg or greater, about 70 mg or greater, about 100 mg or greater, about 150 mg or greater, about 200 mg or greater, about 300 mg or greater, about 400 mg or greater, about 500 mg or greater, or about 700 mg or greater, and the upper limit of the content may be, for example, about 1000 mg or lower, about 800 mg or lower, about 700 mg or lower, about 600 mg or lower, about 500 mg or lower, about 400 mg or lower, about 300 mg or lower, about 200 mg or lower, or about 100 mg or lower. The content may be any range of numerical values between these lower limits and upper limits.

In one embodiment of the present disclosure, the composition of the present disclosure can comprise any additive or any component that can be used for a composition that can be ingested in a body other than GABA in accordance with the form thereof. Examples of these additives and components include, but are not limited to, vitamins including vitamin E and vitamin C, minerals, nutrient components, bioactive components such as aromatics, excipients mixed upon preparation, binders, emulsifiers, tensioning agents (isotonicifier), buffers, dissolution assisting agents, antiseptic agents, stabilizers, antioxidants, coloring agents, coagulants, or coating agents and the like.

In one embodiment of the present disclosure, the form of the composition of the present disclosure is not particularly limited as long as the composition includes GABA, wherein the composition can be, for example, a pharmaceutical composition, drinking/eating product (including food for specified health, nutrition function food, functional food such as food with a functional claim, health assistant food, health food, supplement and the like), food additive, quasi-drug, or medicament.

In one embodiment of the present disclosure, the composition of the present disclosure can be orally ingested wherein when used as an oral agent, the form thereof can be, for example, a tablet (including coated tablet), capsule, powder agent, granular agent, powdered agent, liquid agent, suspension, emulsion, particle-like agent, powder agent, round agent, paste-like agent, cream-like agent, couplet-like agent, gel-like agent, chewable agent, stick-like agent, or the like. In addition, the composition can be used as a raw material of other medicaments after being regulated to a form that can easily be mixed such as powder form or granule form.

In one embodiment of the present disclosure, the composition of the present disclosure can be used for non-therapeutic purposes, which can be a non-therapeutic method of suppressing reduction in cognitive function and/or improving the cognitive function by ingesting the composition of the present disclosure. Such a method does not include therapeutic actions or medical actions and the subject thereof is not particularly limited. The subject is preferably a healthy person, and more preferably the subject scores 24 or higher in the Mini Mental State Examination (MMSE). Reduction in cognitive function can be suppressed and/or the cognitive function can be improved by having such a healthy person ingest the composition of the present disclosure. In addition, when used as a medicament or quasi-drug, it is possible to solicit the efficacy of suppressing reduction in cognitive function and/or improving the cognitive function or the like. MMSE is an examination for assessing the degree of progression of dementia or the like based on the total score with 30 as the perfect score, wherein dementia is suspected with a score of 23 or lower. Thus, by subjecting people who have an MMSE score of 24 or higher and are aware of reduction in cognitive function to ingestion of the composition of the present disclosure, there is a possibility of achieving suppression of reduction in cognitive function and/or improvement in the cognitive function in people who have not reached dementia but are more and more forgetful in their normal lives, or have mild cognitive disorder which is considered potential dementia.

In one embodiment of the present disclosure, the amount of ingestion per day of the GABA comprised in the composition of the present disclosure can be appropriately regulated based on the form of the composition, method of ingestion, purpose of use and the ingestion subject's age, weight, symptom and the like. For example, from the viewpoint of more significantly exerting the effect of GABA comprised in the composition of the present disclosure, the amount of ingestion per day of the GABA comprised in the composition of the present disclosure preferably can be the ingestion that would achieve about 10 mg/day or greater, more preferably can be the ingestion that achieves about 25 mg/day or greater, about 50 mg/day or greater, about 60 mg/day or greater, about 80 mg/day or greater, about 100 mg/day or greater, about 120 mg/day or greater, about 150 mg/day or greater, about 180 mg/day or greater, about 200 mg/day or greater, about 300 mg/day or greater, about 400 mg/day or greater, about 500 mg/day or greater, about 800 mg/day or greater, or about 1000 mg/day or greater. The upper limit of the amount of ingestion per day of the GABA comprised in the composition of the present disclosure is not particularly limited as long as the amount is in the range in which the composition of the present disclosure can exert the effect of suppressing reduction in cognitive function and/or improving the cognitive function.

In one embodiment of the present disclosure, the frequency of application of the method of the present disclosure, or the frequency of ingestion of the composition of the present disclosure may be once or divided into multiple times in one day within the range of the desired amount of ingestion. In addition, the ingestion period can be appropriately set in the range in which the composition of the present disclosure can exert the effect of suppressing reduction in cognitive function and/or improving the cognitive function.

Herein, “about” refers to ±10% of the numerical value that follows.

### EXAMPLES

While the present disclosure is explained in more details below using Examples, the present disclosure is not limited to these Examples.

The experimentation methods and materials used in the present disclosure are explained below. While the present embodiment uses the following experimentation method, the same result can be obtained by using other experimentation methods.

In the following Examples, a test food comprising GABA has been clinically tested to confirm the effect of improving cognitive function and improving health-associated QOL in a human who ingested the test food and has been tested regarding IGF-1 concentration in blood of a human who ingested the test food.

### Example 1

Two types of placebo-controlled randomized double-blind two-group parallel comparison tests were carried out as clinical tests. Regarding the test food, capsules comprising 100 mg of GABA per capsule were used as an active food. In addition, capsules not comprising GABA were used as a placebo food (comparison food). Specifically, capsules comprising 100 mg of dextrin per capsule were used as the placebo food.

Upon selecting subjects, people who fall under all of the eligibility criteria (1) to (5) and do not fall under all of the exclusion criteria (1) to (7) have been selected by screening examination. The screening examination was carried out to select 60 subjects for each test (a total of 120 subjects). MMSE (Mini Mental State Examination) is an examination for assessing the degree of progression of dementia or the like based on the score thereof with 30 as the perfect score, wherein dementia is suspected with a score of 23 or lower.

### <Eligibility Criteria>

(1) Japanese male and female at the age of 40 or higher
(2) People aware of reduction in cognitive function
(3) People who can continuously ingest the test food every day for 12 weeks
(4) People with an MMSE score of 24 or higher
(5) People who have received sufficient explanation of the purpose/content of the clinical test, have the ability to give consent, voluntarily applied to participate in the clinical test with sufficient understanding, and gave consent to the participation in the clinical test on paper

### <Exclusion Criteria>

(1) People who are taking some sort of medication, receiving outpatient therapy, or exercise or diet therapy under the management of a physician
(2) People who are receiving outpatient therapy for mental disorder, sleeping disorder, drug addiction, alcohol addiction, or the like, or have a history of mental disease
(3) People who work night shifts or have alternative work schedules
(4) People who currently have, or have a history of, a serious disease such as diabetes, liver disease, kidney disease, or heart disease, thyroid disease, adrenal gland disease, or other metabolic diseases
(5) People taking health food, supplement and medicament that affects cognitive function
(6) People who cannot observe the management items during the clinical test period
(7) Other people who have been determined as having a problem upon performing the test

The total of 120 subjects were randomly divided into test A active group, test A placebo group, test B active group and test B placebo group with 30 subjects in each group. Each subject of the test A active group ingested two capsules of the active food, each subject of the test A placebo group ingested two capsules of the placebo food, each subject of the test B active group ingested one capsule of the active food and each subject of the test B placebo group ingested one capsule of the placebo food.

Each subject followed the schedule shown in the table below and ingested one or two of the assigned capsules together with water for 12 weeks from two weeks after the initial screening examination. All subjects had no particular instruction during the ingestion period and freely took their meals. In test A, all subjects in the GABA group and the placebo group completed the test. Meanwhile, in test B, two subjects of the placebo group fell out for personal reasons and the number of final subjects to be analyzed were 30 subjects in the GABA group and 28 subjects in the placebo group.

TABLE 1

| | | Measure- | | Measure- | Measure- | Measure- | Measure- |
|---|---|---|---|---|---|---|---|
| | | ment | | ment | ment | ment | ment |
| | | before | | after four | after eight | after twelve | four weeks |
| | | starting | Start | weeks of | weeks of | weeks of | after ending |
| Item | Screening | ingestion | ingestion | ingestion | ingestin | ingestion | ingestion |
| Life | | [symbol] | | | | | |
| questionnaire | | | | | | | |
| Test food | | | [symbol] | | | | |
| ingestion | | | | | | | |
| Inquiry | ● | ● | | ● | ● | ● | ● |
| Physical | ● | ● | | ● | ● | ● | ● |
| examination | | | | | | | |
| MMSE | ● | | | | | | |
| Cognitrax | ● | ● | | ● | ● | ● | |
| RBANS | | ● | | | | ● | |
| SF-3 [symbol] | | ● | | ● | ● | ● | |
| Blood test | ● | ● | | ● | ● | ● | ● |
| Urine test | ● | ● | | ● | ● | ● | ● |

[symbol]

indicates data missing or illegible when filed

As examinations for confirming the effect of improving cognitive function, Cognitrax test and RBANS neuropsychological test were performed. As shown with ● in the test schedule in Table 1, Cognitrax was carried out a total of five times, which are upon screening, before starting test food sample ingestion, after four weeks of ingestion, after eight weeks of ingestion and after twelve weeks of ingestion, and the RBANS neuropsychological test was carried out a total of two times, which are before staring test food ingestion and after twelve weeks of ingestion.

In addition, SF-36 was practiced as health-associated QOL assessment. Regarding SF-36, examination was carried out a total of four times, which are before starting test food ingestion, after four weeks of ingestion, after eight weeks of ingestion and after twelve weeks of ingestion.

(Cognitrax Test)

Tables 2 and 3 show the examination results of 16 domains that can be measured by the Cognitrax test. The result of test A (ingest 200 mg of active food or placebo food) is shown in Table 2 and the result of test B (ingest 100 mg of active food or placebo food) is shown in Table 3.

In addition, the differences between the group of active food and the group of placebo food in test A and test B are shown in Tables 4 and 5, respectively.

TABLE 2
200 mg Ingestion test

| | Group | Before ingestion | 4 w | 8 w | 12 w |
|---|---|---|---|---|---|
| Neurocognition | GABA | 104.3 ± 9.4 | 105.6 ± 7.6 | 106.1 ± 9.9 | 107.2 ± 7.3 |
| index | Placebo | 99.2 ± 12.0 | 102.7 ± 7.5 | 104.5 ± 10.0 | 105.2 ± 9.1 |
| Composite | GABA | 101.9 ± 16.8 | 100.5 ± 13.1 | 103.5 ± 12.4 | 100.9 ± 16.0 |
| memory | Placebo | 96.3 ± 21.8 | 101.4 ± 15.1 | 106.4 ± 14.8 | 105.9 ± 14.0 |
| Verbal | GABA | 102.1 ± 17.5 | 103.6 ± 13.9 | 104.9 ± 13.1 | 105.3 ± 14.2 |
| memory | Placebo | 97.6 ± 18.9 | 103.4 ± 16.7 | 111.2 ± 14.3 | 108.4 ± 13.7 |
| Visual | GABA | 101.0 ± 14.0 | 97.7 ± 10.9 | 100.7 ± 12.4 | 96.7 ± 15.5 |
| memory | Placebo | 96.9 ± 20.1 | 99.1 ± 13.5 | 99.4 ± 12.8 | 101.4 ± 15.6 |
| Psychomotor | GABA | 114.6 ± 17.7 | 116.9 ± 18.4 | 115.7 ± 19.5 | 118.1 ± 16.1 |
| speed | Placebo | 107.5 ± 14.7 | 109.9 ± 12.7 | 110.6 ± 14.0 | 111.7 ± 13.3 |
| Reaction | GABA | 96.6 ± 14.5 | 99.3 ± 0.4 | 94.0 ± 21.9 | 100.7 ± 10.4 |
| time | Placebo | 92.1 ± 16.7 | 94.3 ± 11.9 | 94.6 ± 15.3 | 95.2 ± 12.3 |
| Complex | GABA | 105.6 ± 9.8 | 106.6 ± 9.3 | 108.8 ± 8.5 | 109.0 ± 7.4 |
| attention | Placebo | 102.2 ± 11.7 | 106.0 ± 8.8 | 107.5 ± 9.7 | 107.3 ± 11.3 |
| Cognitive | GABA | 102.5 ± 9.4 | 104.4 ± 9.8 | 108.2 ± 8.6 | 107.5 ± 7.3 |
| flexibility | Placebo | 98.1 ± 11.8 | 102.2 ± 10.8 | 103.6 ± 10.9 | 106.1 ± 7.9 |
| Processing | GABA | 118.2 ± 13.4 | 121.2 ± 13.7 | 122.4 ± 14.9 | 123.1 ± 12.4 |
| speed | Placebo | 114.6 ± 12.9 | 117.1 ± 11.3 | 118.9 ± 13.8 | 120.0 ± 11.3 |
| Executive | GABA | 102.8 ± 9.0 | 104.5 ± 9.8 | 108.5 ± 8.6 | 107.4 ± 7.1 |
| function | Placebo | 98.3 ± 11.7 | 102.1 ± 11.1 | 103.6 ± 10.5 | 106.0 ± 8.1 |
| Social | GABA | 87.9 ± 17.9 | 93.2 ± 18.9 | 91.3 ± 20.8 | 89.0 ± 23.1 |
| acuity | Placebo | 94.3 ± 18.1 | 95.4 ± 17.4 | 91.6 ± 18.5 | 96.9 ± 15.8 |
| Non-verbal | GABA | 102.4 ± 14.9 | 102.6 ± 9.1 | 104.9 ± 12.5 | 107.0 ± 10.2 ** |
| reasoning | Placebo | 98.7 ± 11.6 | 98.4 ± 8.6 | 107.1 ± 8.8 | 100.1 ± 9.5 |
| Working | GABA | 107.0 ± 12.2 | 108.5 ± 11.2 | 113.4 ± 9.7 ** | 111.2 ± 11.5 |
| memory | Placebo | 103.5 ± 12.2 | 105.5 ± 12.2 | 106.2 ± 11.0 | 107.0 ± 11.7 |
| Continuous | GABA | 109.9 ± 10.9 | 109.0 ± 12.4 | 114.3 ± 8.2 * | 112.9 ± 8.2 |
| attention | Placebo | 106.6 ± 10.7 | 108.0 ± 10.3 | 108.7 ± 8.6 | 109.2 ± 8.1 |
| Simple | GABA | 97.9 ± 17.4 | 100.2 ± 10.5 | 99.2 ± 14.9 | 97.9 ± 17.0 |
| attention | Placebo | 95.7 ± 19.9 | 99.3 ± 11.5 | 99.5 ± 13.1 | 92.4 ± 29.5 |
| Motor | GABA | 107.6 ± 17 | 108.4 ± 18.3 | 105.8 ± 20.1 | 108.7 ± 17.7 |
| speed | Placebo | 100.9 ± 14.3 | 102.5 ± 12.9 | 101.9 ± 13.2 | 102.6 ± 13.7 |

Values represent mean ± SD.
* p < 0.05.
** p < 0.01 vs. Placebo as determined using unpaired t-tests.

TABLE 3
100 mg Ingestion test

| | | Beofre | | | |
|---|---|---|---|---|---|
| | Group | ingestion | 4 w | 8 w | 12 w |
| Neurocognition | GABA | 106.4 ± 6.7 | 106.8 ± 7.6 | 108.3 ± 8.1 | 108.5 ± 7.3 |
| index | Placebo | 103.0 ± 11.6 | 105.8 ± 10.5 | 105.5 ± 11.5 | 107.7 ± 7.41 |
| Composite | GABA | 105.4 ± 13.4 | 105.8 ± 14.6 | 107.2 ± 14.3 | 107.4 ± 15.5 |
| memory | Placebo | 99.9 ± 18.0 | 98.6 ± 15.0 | 99.8 ± 16.2 | 101.8 ± 13.3 |
| Verbal | GABA | 107.2 ± 12.6 | 109.1 ± 15.0 | 108.7 ± 15.1 | 111.4 ± 14.1 |
| memory | Placebo | 100.8 ± 18.6 | 100.0 ± 19.5 | 99.9 ± 21.3 | 104.6 ± 16.3 |
| Visual | GABA | 101.2 ± 15.1 | 100.2 ± 15.1 | 103.0 ± 12.5 | 100.6 ± 14.9 |
| memory | Placebo | 99.2 ± 15.4 | 97.8 ± 10.8 | 99.8 ± 11.0 | 98.5 ± 13.5 |
| Psychomotor | GABA | 114.3 ± 12.6 | 118.2 ± 14.3 | 117.7 ± 13.1 | 119.2 ± 12.0 |
| speed | Placebo | 109.8 ± 12.1 | 112.8 ± 12.7 | 111.5 ± 14.2 | 113.8 ± 11.4 |
| Reaction | GABA | 98.1 ± 9.0 | 97.8 ± 10.8 | 97.9 ± 10.0 | 96.6 ± 10.7 |
| time | Placebo | 96.2 ± 13.8 | 97.0 ± 17.7 | 96.4 ± 18.6 | 98.3 ± 12.5 |
| Complex | GABA | 108.8 ± 8.4 | 106.3 ± 12.9 | 109.6 ± 10.3 | 109.9 ± 10.1 |
| attention | Placebo | 105.8 ± 12.8 | 110.4 ± 7.2 | 110.0 ± 9.4 | 112.4 ± 5.4 |
| Cognitive | GABA | 105.9 ± 10.0 | 105.8 ± 11.3 | 109.1 ± 12.3 | 109.4 ± 10.7 |
| flexibility | Placebo | 103.5 ± 12.5 | 109.4 ± 10.7 | 109.6 ± 11.7 | 111.7 ± 8.9 |
| Processing | GABA | 117.1 ± 11.2 | 122.8 ± 10.7 | 124.6 ± 13.7 | 126.0 ± 13.0 |
| speed | Placebo | 118.7 ± 13.6 | 122.0 ± 15.4 | 123.1 ± 15.9 | 123.6 ± 17.2 |
| Executive | GABA | 105.5 ± 9.9 | 105.9 ± 10.9 | 109.0 ± 12.3 | 109.0 ± 10.7 |
| function | Placebo | 103.3 ± 12.6 | 109.2 ± 11.0 | 109.4 ± 11.7 | 111.4 ± 9.1 |
| Social | GABA | 92.6 ± 16.0 | 90.5 ± 16.7 | 95.4 ± 17.0 | 97.2 ± 17.0 |
| acuity | Placebo | 91.8 ± 18.7 | 94.9 ± 16.7 | 89.4 ± 22.2 | 92.6 ± 24.8 |
| Non-verbal | GABA | 102.6 ± 14.1 | 104.9 ± 11.7 | 106.2 ± 12.2 | 107.5 ± 10.6 |
| reasoning | Placebo | 99.5 ± 14.6 | 104.4 ± 11.6 | 105.7 ± 12.8 | 103.6 ± 12.7 |
| Working | GABA | 110.5 ± 7.9 | 110.4 ± 9.3 | 109.8 ± 9.5 | 112.2 ± 7.9 |
| memory | Placebo | 107.9 ± 10.9 | 110.0 ± 9.3 | 111.7 ± 8.4 | 111.3 ± 8.2 |
| Continuous | GABA | 111.6 ± 6.9 | 111.8 ± 7.2 | 110.2 ± 8.9 | 110.8 ± 13.8 |
| attention | Placebo | 110.4 ± 7.7 | 112.4 ± 6.2 | 111.0 ± 8.3 | 110.8 ± 9.3 |
| Simple | GABA | 101.2 ± 14.9 | 97.8 ± 25.2 | 101.3 ± 13.0 | 99.2 ± 21.3 |
| attention | Placebo | 97.9 ± 19.4 | 103.5 ± 6.9 | 98.5 ± 15.2 | 105.7 ± 4.2 |
| Motor | GABA | 107.2 ± 13.3 | 109.0 ± 15.1 | 107.2 ± 13.9 * | 108.3 ± 12.9 * |
| speed | Placebo | 100.8 ± 12.2 | 102.1 ± 11.4 | 99.8 ± 12.5 | 101.8 ± 11.1 |

Values represent mean ± SD.
* p < 0.05 vs. Placebo as determines using unpaired t-tests.

TABLE 4

| | Beofre | | | |
|---|---|---|---|---|
| | ingestion | 4 w | 8 w | 12 w |
| Neurocognition index | 5.1 | 2.9 | 1.6 | 2.1 |
| Composite memory | 5.6 | −0.9 | −2.9 | −5.0 |
| Verbal memory | 4.6 | 0.2 | −6.3 | −3.1 |
| Visual memory | 4.0 | −1.4 | 1.4 | −4.6 |
| Psychomotor speed | 7.1 | 7.1 | 5.2 | 6.4 |
| Reaction time | 4.5 | 5.0 | −0.6 | 5.5 |
| Complex attention | 3.4 | 0.6 | 1.3 | 1.7 |
| Cognition flexibility | 4.4 | 2.1 | 4.6 | 1.4 |
| Processing speed | 3.6 | 4.1 | 3.5 | 3.1 |
| Executive function | 4.5 | 2.4 | 4.9 | 1.4 |
| Social acuity | −6.4 | −2.2 | −0.3 | −8.0 |
| Non-verbal reasoning | 3.7 | 4.2 | −2.2 | 6.8 |
| Working memory | 3.5 | 3.0 | 7.2 | 4.2 |
| Continuous attention | 3.3 | 1.1 | 5.6 | 3.7 |
| Simple attention | 2.2 | 0.9 | −0.3 | 5.5 |
| Motor spee | 6.7 | 6.0 | 3.9 | 6.1 |

TABLE 5

| | Brofre | | | |
|---|---|---|---|---|
| | ingestion | 4 w | 8 w | 12 w |
| Neurocognitive index | 3.4 | 1.0 | 2.9 | 0.8 |
| Composite memory | 5.2 | 7.2 | 7.4 | 5.5 |
| Verbal memory | 6.4 | 9.1 | 8.8 | 6.8 |
| Visual memory | 2.0 | 2.4 | 3.1 | 2.1 |
| Psychomotor speed | 4.5 | 5.3 | 6.3 | 5.4 |
| Reaction time | 1.9 | 0.8 | 1.4 | −1.7 |
| Complex attention | 3.0 | −4.0 | −0.4 | −2.5 |
| Cognition flexibility | 2.4 | −3.6 | −0.5 | −2.3 |
| Processing speed | 1.5 | 0.8 | 1.5 | 2.4 |
| Executive function | 2.1 | −3.2 | −0.4 | −2.4 |
| Social acuity | 0.8 | −4.4 | 6.1 | 4.6 |
| Non-verbal reasoning | 3.1 | 0.5 | 0.5 | 3.9 |
| Working memory | 2.6 | 0.4 | −1.8 | 0.9 |
| Continuous attention | 1.2 | −0.6 | −0.8 | 0.0 |
| Simple attention | 3.3 | −5.8 | 2.8 | −6.5 |
| Motor speed | 6.4 | 6.9 | 7.3 | 6.5 |

According to the result in Table 2, in test A, significant improvement was able to be confirmed in the scores regarding working memory and continuous attention in the GABA group compared to the placebo group after eight weeks of ingestion, and significant improvement was able to be confirmed in the scores regarding reasoning in the GABA group compared to the placebo group after twelve weeks of ingestion. In addition, according to the result in Table 3, in test B, significant improvement was able to be confirmed in the scores regarding motor speed in the GABA group compared to the placebo group at least after eight weeks of ingestion. It can be understood from the above that GABA is effective in improving reasoning, working memory, continuous attention and motor speed of a healthy person.

Among the above, the result of improvement in reasoning is surprising. In the subject technical field, reasoning and other brain functions are not necessarily associated, and improvement in brain functions other than reasoning was not necessarily considered to lead to improvement in reasoning. For example, while it was considered that high concentration of lutein or zeaxanthin in blood serum or the central nervous system and an improvement in cognitive function of elderly people are associated, it has been reported that there is no improvement in reasoning, which was measured by the CNS Vital Signs examination method which forms the basis of Cognitrax, even upon continuous ingestion of lutein or zeaxanthin for a year (Nutrients. 2017 Nov. 14; 9(11)). In addition, while aerobic exercise is used as a part of rehabilitation for concussions, it has been reported that aerobic exercise does not improve reasoning by the CNS Vital Signs examination method (J Athl Train. 2018 December; 53(12):1156-1165). Therefore, improvement in reasoning along with other brain functions such as working memory or continuous attention by continuous ingestion of GABA disproves the conventional recognition.

(RBANS Neuropsychological Test)

Tables 6 and 7 show the examination results regarding five domains that can be measured by the RBANS neuropsychological test. The result of test A (ingest 200 mg of active food or placebo food) is shown in Table 6 and the result of test B (ingest 100 mg of active food or placebo food) is shown in Table 7. In addition, the differences between the group of active food and the group of placebo food in test A and test B are shown in Tables 8 and 9, respectively.

TABLE 6
200 Ingestion test

| | Group | Before ingestion | 12 w |
|---|---|---|---|
| Immediate | GABA | 42.3 ± 16.6 | 47.6 ± 14.7 |
| memory | Placebo | 43.1 ± 12.6 | 42.0 ± 12.2 |
| Visual spatial | GABA | 47.7 ± 14.7 | 48.8 ± 13.0 ** |
| configuration | Placebo | 46.9 ± 11.7 | 35.7 ± 15.6 |
| Language | GABA | 48.4 ± 8.5 | 54.2 ± 9.5 |
| | Placebo | 48.5 ± 9.7 | 51.1 ± 11.6 |
| Attention | GABA | 44.5 ± 17.6 | 53.0 ± 15.2 |
| | Placebo | 42.2 ± 14.7 | 48.0 ± 12.6 |
| Delayed | GABA | 39.8 ± 17.1 | 46.0 ± 15.5 * |
| memory | Placebo | 41.4 ± 15.5 | 36.6 ± 12.2 |

Values represent mean ± SD.
* p < 0.05.
** p < 0.01 vs. Placebo as determined using unpaired t-tests.

TABLE 7
100 mg Ingestion test

| | Group | Before ingestion | 12 w |
|---|---|---|---|
| Immediate | GABA | 42.8 ± 13.4 | 43.9 ± 14.4 |
| memory | Placebo | 43.7 ± 15.7 | 40.4 ± 14.4 |
| Visual spatial | GABA | 48.3 ± 8.8 | 48.2 ± 10.3 ** |
| configuration | Placebo | 47.4 ± 14.4 | 38.3 ± 14.7 |
| Language | GABA | 47.8 ± 11.6 | 51.6 ± 8.7 |
| | Placebo | 49.8 ± 11.4 | 47.7 ± 8.6 |
| Attention | GABA | 43.4 ± 11.6 | 54.0 ± 11.5 |
| | Placebo | 48.9 ± 14.5 | 56.1 ± 15.6 |
| Delayed | GABA | 41.7 ± 15.3 | 46.9 ± 11.3 ** |
| memory | Placebo | 40.5 ± 19.0 | 35.0 ± 16.3 |

Values represent mean ± SD.
** p < 0.01 vs. Placebo as determines using unpaired t-tests.

TABLE 8

| | Before ingestion | 12 w |
|---|---|---|
| | −0.8 | 5.6 |
| Immediate memory | 0.8 | 13.1 |
| Visual space | −0.2 | 3.1 |
| Language | 2.3 | 5.0 |
| Delayed memory | −1.6 | 9.4 |

TABLE 9

| | Before ingestion | 12 w |
|---|---|---|
| Immediate memory | −0.9 | 3.5 |
| Visual space | 1.0 | 9.9 |
| Language | −2.1 | 3.9 |
| Attention | −5.5 | −2.2 |
| Delayed memory | 1.2 | 11.9 |

According to the results in Tables 6 and 7, in both tests A and B, significant improvement was able to be confirmed in the scores regarding visual space and delayed playback in the GABA group compared to the placebo group after twelve weeks of ingestion. It can be understood from the above that GABA is effective in improving visual space and delayed playback of a healthy person.

(SF-36)

Tables 10 and 11 show the examination results regarding eight domains that can be measured by SF-36. The result of test A (ingest 200 mg of active food or placebo food) is shown in Table 10 and the result of test B (ingest 100 mg of active food or placebo food) is shown in Table 11. In addition, the differences between the group of active food and the group of placebo food in test A and test B are shown in Tables 12 and 13, respectively.

TABLE 10
200 mg Ingestion test

| | | Before | | | |
|---|---|---|---|---|---|
| | Group | ingestion | 4 w | 8 w | 12 w |
| Physical | GABA | 53.0 ± 6.1 | 53.8 ± 6.2 | 53.8 ± 6.1 | 53.6 ± 7.2 |
| function | Placebo | 52.6 ± 5.2 | 53.4 ± 5.6 | 54.5 ± 4.5 | 54.6 ± 4.6 |
| Everyday role | GABA | 54.2 ± 4.3 | 54.3 ± 3.5 | 53.8 ± 4.0 * | 54.2 ± 4.2 |
| function (physical) | Placebo | 52.8 ± 5.9 | 51.5 ± 7.8 | 50.5 ± 6.7 | 52.1 ± 6.7 |
| Body | GABA | 53.9 ± 7.9 | 54.4 ± 8.1 | 53.0 ± 9.2 | 55.1 ± 8.5 |
| pain | Placebo | 54.6 ± 7.5 | 56.5 ± 6.2 | 55.9 ± 7.7 | 55.4 ± 7.6 |
| General | GABA | 57.4 ± 7.4 | 57.2 ± 7.9 | 58.4 ± 7.4 | 58.4 ± 6.5 |
| health | Placebo | 57.4 ± 6.3 | 57.2 ± 6.4 | 55.9 ± 7.2 | 58.2 ± 7.9 |
| Vitality | GABA | 54.6 ± 7.6 | 56.1 ± 7.3 ** | 55.6 ± 8.3 * | 57.1 ± 6.7 * |
| | Placebo | 51.8 ± 7.4 | 50.7 ± 7.8 | 50.8 ± 6.7 | 51.8 ± 8.9 |
| Social | GABA | 54.4 ± 5.2 | 54.9 ± 5.2 | 53.4 ± 7.7 | 53.4 ± 8.1 |
| life | Placebo | 52.3 ± 7.9 | 53.8 ± 5.8 | 52.5 ± 8.0 | 54.4 ± 5.0 |
| Everyday role | GABA | 52.6 ± 6.3 | 53.4 ± 6.0 | 52.7 ± 5.7 | 53.7 ± 5.4 |
| function (mental) | Placebo | 52.7 ± 5.1 | 51.5 ± 7.5 | 51.6 ± 6.2 | 52.2 ± 7.1 |
| Mental | GABA | 54.1 ± 8.4 | 56.0 ± 6.7 * | 56.3 ± 7.3 | 57.0 ± 5.9 |
| health | Placebo | 53.8 ± 7.2 | 52.4 ± 7.0 | 52.8 ± 8.0 | 53.5 ± 8.4 |

Values represent mean ± SD.
* p < 0.05.
** p < 0.01 vs. Placebo as determined using unpaired t-tests.

TABLE 11
100 mg Ingestion test

| | Group | Before ingestion | 4 w | 8 w | 12 w |
|---|---|---|---|---|---|
| Physical | GABA | 55.6 ± 3.5 | 55.2 ± 3.7 | 56.6 ± 2.4 * | 55.9 ± 3.1 |
| function | Placebo | 52.6 ± 5.2 | 53.4 ± 5.6 | 54.5 ± 4.5 | 54.6 ± 4.6 |
| Everyday role | GABA | 53.5 ± 3.9 | 53.8 ± 4.2 | 53.9 ± 3.5 | 53.1 ± 4.2 |
| function (physical) | Placebo | 52.8 ± 5.9 | 51.5 ± 7.8 | 50.5 ± 6.7 | 52.1 ± 6.7 |
| Body | GABA | 55.9 ± 5.8 | 56.2 ± 6.7 | 56.8 ± 6.4 | 57.1 ± 5.5 |
| pain | Placebo | 54.6 ± 7.5 | 56.5 ± 6.2 | 55.9 ± 7.7 | 55.4 ± 7.6 |
| General | GABA | 57.5 ± 7.1 | 58.3 ± 7.4 | 57.2 ± 7.5 | 59.2 ± 6.2 |
| health | Placebo | 57.4 ± 6.3 | 57.2 ± 6.4 | 55.9 ± 7.2 | 58.2 ± 7.9 |
| Vitality | GABA | 54.4 ± 7.6 | 53.7 ± 7.6 | 54.8 ± 7.2 | 54.5 ± 7.4 |
| | Placebo | 51.8 ± 7.4 | 50.7 ± 7.8 | 50.8 ± 6.7 | 51.8 ± 8.9 |
| Social life | GABA | 55.9 ± 3.0 | 54.7 ± 5.2 | 55.7 ± 3.1 | 55.1 ± 4.5 |
| function | Placebo | 52.3 ± 7.9 | 53.8 ± 5.8 | 52.5 ± 8.0 | 54.4 ± 5.0 |
| Everyday role | GABA | 53.7 ± 4.7 | 53.0 ± 5.5 | 53.7 ± 3.9 | 53.9 ± 4.3 |
| function (mental) | Placebo | 52.7 ± 5.1 | 51.5 ± 7.5 | 51.6 ± 6.2 | 52.2 ± 7.1 |
| Mental | GABA | 56.5 ± 5.4 | 55.8 ± 5.8 | 56.1 ± 5.8 | 56.4 ± 5.6 |
| health | Placebo | 33.8 ± 7.2 | 52.4 ± 7.0 | 52.8 ± 8.0 | 53.5 ± 8.4 |

Values represent mean ± SD.
* p < 0.05 vs. Placebo as determined using unpaired t-tests.

TABLE 12

| | Before ingestion | 4 W | 8 W | 12 W |
|---|---|---|---|---|
| Physical function | 0.5 | 0.4 | −0.7 | −1.0 |
| Everyday role | 1.3 | 2.8 | 3.3 | 2.1 |
| function (physical) | | | | |
| Body pain | −0.7 | −2.1 | −2.9 | −0.3 |
| General health | 0.0 | 0.1 | 2.5 | 0.2 |
| Vitality | 2.9 | 5.5 | 4.8 | 5.4 |
| Social life function | 2.1 | 1.1 | 0.9 | −1.1 |
| Everyday role | −0.1 | 1.9 | 1.1 | 1.5 |
| function (mental) | | | | |
| Mental health | 0.3 | 3.6 | 3.5 | 3.5 |

TABLE 13

| | Before ingestion | 4 W | 8 W | 12 W |
|---|---|---|---|---|
| Physical function | 3.0 | 1.8 | 2.2 | 1.3 |
| Everyday role | 0.7 | 2.3 | 3.4 | 1.0 |
| function (physical) | | | | |
| Body pain | 1.3 | −0.3 | 0.9 | 1.8 |
| General health | 0.1 | 1.2 | 1.2 | 1.0 |
| Vitality | 2.7 | 3.0 | 4.0 | 2.8 |
| Social life function | 3.7 | 0.9 | 3.2 | 0.6 |
| Everyday role | 1.0 | 1.5 | 2.1 | 1.7 |
| function (mental) | | | | |
| Mental health | 2.7 | 3.3 | 3.3 | 2.9 |

According to the result in Table 11, in test A, significant improvement was able to be confirmed in the scores regarding energy in the GABA group compared to the placebo group at least after four weeks of ingestion. Furthermore, significant improvement was able to be confirmed in the scores regarding emotional health after four weeks of ingestion and regarding everyday role function (physical) after eight weeks of ingestion in the GABA group compared to the placebo group. In addition, according to the result in Table 12, in test B, significant improvement was able to be confirmed in the scores regarding physical function in the GABA group compared to the placebo group after eight weeks of ingestion. It can be understood from the above that GABA is effective in improving everyday role function (physical), energy, emotional health and physical function of a healthy person.

### Example 2

Among the 120 subjects in Example 1, a total of six blood tests were carried out to the total of 60 subjects who participated in test, wherein blood tests were carried out upon screening, before starting test food ingestion, after four weeks of ingestion, after eight weeks of ingestion, after twelve weeks of ingestion and four weeks after ending ingestion, as shown with ● in the test schedule in Table 1. The result thereof is shown in Table 14.

TABLE 14
200 mg Ingestion test

| | Group | Before ingestion | 4 w | 8 w | 12 w |
|---|---|---|---|---|---|
| IGF-1 | GABA | 71.0 ± 21.5 | 75.3 ± 21.3 | 77.2 ± 18.9 | 83.9 ± 26.3 † |
| (ng/mL) | Placebo | 83.6 ± 30.0 | 86.9 ± 31.0 | 82.2 ± 26.5 | 86.9 ± 24.1 |

Values represent mean ± SD.
† p < 0.1 vs. before ingestion as determined using Dunnett's tests.

As shown in Table 14, in the group that ingested GABA, the tendency of increase in IGF-1 was able to be confirmed. It can be understood from the above that GABA increases IGF-1 of a healthy person, contributing to improvement in cognitive function.

In addition, it is needless say that the present disclosure can be modified in various ways, and the present disclosure is not limited to the embodiment discussed above. The present disclosure can be modified in various ways within the scope that does not change the gist of the invention.

1. A composition for suppressing reduction in cognitive function and/or improving the cognitive function in a subject, comprising γ-aminobutyric acid.

2. The composition of claim 1, wherein the cognitive function comprises reasoning.

3. The composition of claim 1, wherein the cognitive function comprises working memory.

4. The composition of claim 1, wherein the cognitive function comprises sustained attention.

5. The composition of claim 2, wherein the reasoning, the working memory, or the sustained attention is measured by Cognitrax.

6. The composition of claim 1, wherein the cognitive function comprises spatial cognition.

7. The composition of claim 1, wherein the cognitive function comprises memory.

8. The composition of claim 6, wherein the spatial cognition or the memory is measured by RBANS neuropsychological test.

9. The composition of claim 1, which is for improving physical function.

10. The composition of claim 1, which is for improving vitality.

11. The composition of claim 1, which is for improving mental health.

12. The composition of claim 9, wherein the physical function, the vitality, or the mental health is measured by SF-36.

13. The composition of claim 1, which is for suppressing reduction in cognitive function and/or improving the cognitive function in a subject by increasing an insulin-like growth factor-1 (IGF-1) concentration in blood.

14. The composition of claim 1, wherein a content of the γ-aminobutyric acid is about 10 to about 1000 mg.

15. The composition of claim 1, wherein an ingestion amount of the γ-aminobutyric acid is about 10 to about 1000 mg per day.

16. The composition of claim 1, which is ingested every day for at least four weeks.

17. The composition of claim 1, wherein the subject is a healthy person.

18. The composition of claim 1, wherein the subject has a Mini Mental State Examination (MMSE) score of 24 or higher.

19. The composition of claim 1, which is orally ingested.

20. The composition of claim 1, which is a drinking/eating product, a food additive, a quasi-drug, or a medicament.
